(19) United States
(12) Patent Application Publication
(10) Pub. No.: US 2015/0011951 A1
(43) Pub. Date: Jan. 8, 2015

(54) DRUG DELIVERY SYSTEM USING IMPLANT

(71) Applicants: Chee Il Hwang, Seoul (KR); Shin Jae Lee, Gyeonggi-do (KR); Young Seok Park, Gyeonggi-do (KR)

(72) Inventors: Chee Il Hwang, Seoul (KR); Shin Jae Lee, Gyeonggi-do (KR); Young Seok Park, Gyeonggi-do (KR)

(73) Assignees: SNU R&DB Foundation, Seoul (KR); Chee ll HWANG, Seoul (KR)

(21) Appl. No.: 14/373,018

(22) PCT Filed: Jun. 18, 2012
(86) PCT No.: PCT/KR2012/004795

§ 371 Date: Jul. 17, 2014

(30) Foreign Application Priority Data
Mar. 22, 2012 (KR) 10-2012-0029335

(51) Int. Cl. A61M 31/00

(52) U.S. Cl. CPC A61M 31/002
USPC 604/286

(57) ABSTRACT

The present invention relates to an implant that is implanted into a human body by means of osseointegration. A drug delivery system comprises: an implant (10) for performing a support function, which is implanted into a human body; and a cover unit (20) for performing a covering function, which is coupled to the implant (10). The drug delivery system further comprises: an accommodating unit (12) formed in the implant (10) for accommodating a drug cartridge (30) and intended to be opened/closed by the cover unit (20); a plurality of diffusion units (14) formed in the wall surface of the accommodating unit (12) so as to diffuse and discharge contents of the drug cartridge (30); and injection units (16) formed at the respective ends of the diffusion units (14) for injecting the contents of the drug cartridge (30) into the human body. By continuously injecting drugs using the implant, various inconveniences which might occur due to oral ingestion or injection of the drug and various side effects which might occur due to oral ingestion of the drug are minimized.

### TECHNICAL FIELD

The present invention relates to an implant that is configured to be implanted into the human body by osseointegration, and more particularly, to a drug delivery system using an implant, which continuously can administer a variety of kinds of drugs needed for treatment of a patient's disease into the human body using an implant.

### BACKGROUND ART

In general, a drug delivery system (DDS) is a formulation designed to effectively deliver an appropriately necessary amount of drug by maximizing the efficacy and effectiveness of a drug while minimizing side effects of the drug. The drug delivery system is used as a means that resolves inconvenience involved in oral administration or injection of drug.

For example, in case of the oral administration of an an anti-inflammatory analgesic drug in the treatment of arthritis, the drug delivery system exhibits the effectiveness of drug when the drug acts on a joint, but may also involve the side effects such as occurrence of ulcer in sites other than the joint, particularly, in a gastrointestinal tract. Thus, the drug delivery system is meant to design a formulation to reduce the aforementioned side effects and maximize the effectiveness of drug.

Besides, a novel method is introduced into the delivery path and delivery technique type of drug in view of the medical treatment method, the specific physicochemical characteristics of drug, the pharmacokinetic characteristics, and the like, so that an effective treatment method can be acquired and inconvenience of a patient can be reduced. As such, the drug delivery system is developed and used to give a convenience to a patient and considerable researches thereof are currently in progress.

However, in case of an outpatient with a chronic disease, who requires continuous administration of a drug for a long period of time, the most common administration method is an oral administration of the drug. Thus, there still is a problem in that a patient fails in taking the drug for a long period of time despite excellence in formulation of the drug itself, leading to a serious problem to treatment and suppression of exacerbation of a chronic disease, which results in a considerable loss of costs due to the issues of discarded drugs and the like in terms of personal health as well as national economies.

A medical device called an insulin pump is developed for the treatment of diabetes as one representative one of chronic diseases so that a great medical achievement enabling continuous administration and maintenance of concentration of a drug has been made. However, the insulin pump encounters a problem in that since it employs an injection needle, a patient feels a pain and a feeling of fear and the insulin pump causes exercise limitation in the patient in a daily life.

Besides, a pharmaceutical preparation for treating hypertension, a pharmaceutical preparation for treating Parkinson's disease, and a contraceptive preparation are a kind of drugs which is required to be faithfully taken by patients. However, these preparations has shortcomings in that patients with such diseases forget to take or do not take a drug at the optimum time, resulting in a significant loss of health.

### DISCLOSURE OF INVENTION

### Technical Problem

Accordingly, the present invention has been made to solve the above-mentioned problems occurring in the prior art, and it is an object of the present invention to provide a drug delivery system using an implant, which continuously can administer a variety of kinds of drugs needed for treatment of a patient's chronic disease into the human body using an implant so that inconveniences or side effects involved in regular oral administration and injection of a drug can be minimized.

### Technical Solution

To achieve the above objects, in one aspect, the present invention provides a drug delivery system using an implant including: an implant configured to be implanted into the human body to perform a support function, wherein the implant includes an accommodating part formed therein to allow a drug cartridge to be accommodated therein, a plurality of diffusion parts formed in the circumferential wall of the accommodating part to allow the content of the drug cartridge to be diffusively discharged to the outside of the implant therethrough, and a plurality of injection parts formed at associated ones of distal ends of the diffusion units to allow the content of the drug cartridge to be injected into the human body therethrough; and a cover unit coupled to the implant and configured to perform a closing function to open or close the accommodating part of the implant.

### Advantageous Effects

The drug delivery system using an implant according to the present invention has the following advantageous effects.

First, a drug is continuously administered to a patient by the implant so that various inconveniences and a feeling of fear involved in the long-term oral administration and injection of the drug can be minimized.

Second, a drug is regularly administered to a patient by the implant so that the side effects of the drug, occurring in a gastrointestinal tract during the oral administration of the drug can be minimized.

Third, a drug is directly administered to the human body by the implant so that correctness and rapidness of delivery of the drug are excellent, thereby improving the drug administration efficiency.

Fourth, various workabilities according to the accommodation and replacement of the drug cartridge is improved by the opening/closing action of the cover unit, thereby maximizing a degree of satisfaction of an operator.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a schematic exploded perspective view illustrating a drug delivery system using an implant according to an embodiment of the present invention;

FIGS. 2(a) to 2(c) are cross-sectional views illustrating various modifications of a drug delivery system using an implant according to an embodiment of the present invention; and

FIG. 3 is a cross-sectional view conceptually illustrating the operation state of a drug delivery system using an implant according to an embodiment of the present invention.

### EXPLANATION ON REFERENCE NUMERALS OF MAIN ELEMENTS IN THE DRAWINGS

1: drug delivery device
10: implant
12: accommodating part
12-5: guide groove
14: diffusion part
16: injection part
20: cover unit
30: drug cartridge
32: guide projection
34: withdrawal lug

### BEST MODE FOR CARRYING OUT THE INVENTION

Now, a preferred embodiment of a drug delivery system using an implant according to the present invention will be described hereinafter in detail with reference to the accompanying drawings.

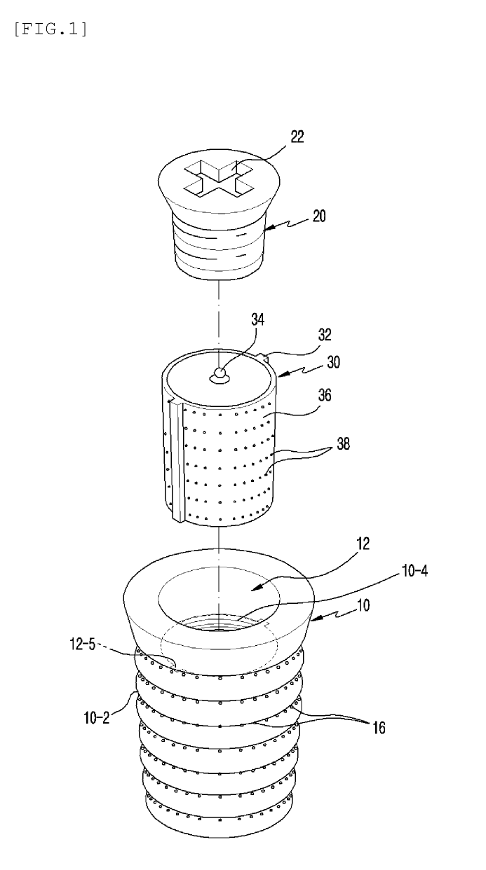

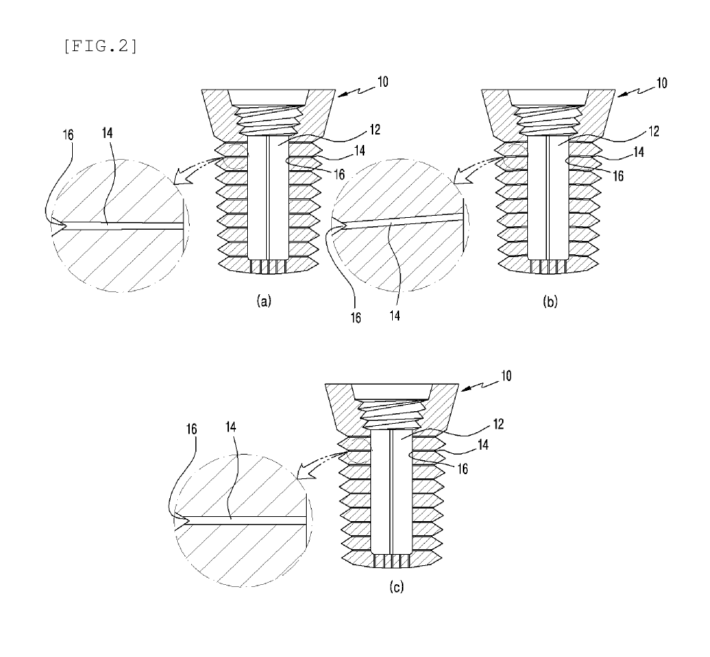

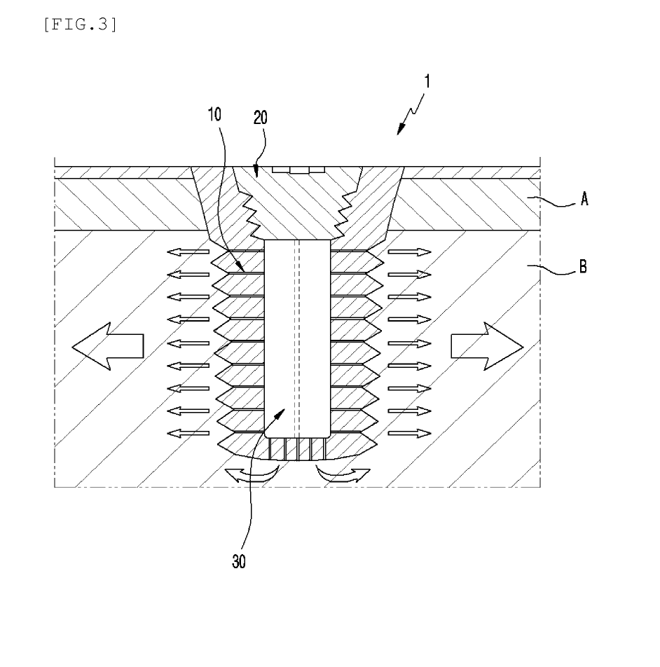

FIG. 1 is a schematic exploded perspective view illustrating a drug delivery system using an implant according to an embodiment of the present invention, FIGS. 2(a) to 2(c) are cross-sectional views illustrating various modifications of a drug delivery system using an implant according to an embodiment of the present invention, and FIG. 3 is a cross-sectional view conceptually illustrating the operation state of a drug delivery system using an implant according to an embodiment of the present invention.

A drug delivery device 1 according to the present invention includes an implant 10 that is implanted into the human body to perform a support and a cover unit 20 that is coupled to the implant 10 to perform a closing function. The implant 10 includes an accommodating part 12 that is formed therein to allow a drug cartridge 30 to be accommodated therein and is opened or closed by the cover unit 20, a plurality of diffusion parts 14 formed in the circumferential wall of the accommodating part 12 to allow the content of the drug cartridge 30 to be diffusively discharged to the outside of the implant therethrough, and a plurality of injection parts 16 formed at associated ones of distal ends of the diffusion units 14 to allow the content of the drug cartridge 30 to be injected into the human body therethrough.

Here, the drug delivery device 1 is configured such that the implant 10, the cover unit 20, and the drug cartridge 30 are coupled to each other. In particular, the implant 10 includes the accommodating part 12, the diffusion part 14, and the injection part 16 so that convenience of administration of a drug into a patient with a chronic disease is enhanced.

In addition, the implant 10 is securely fixed to a body of a patient with a chronic disease, preferably a bone tissue of the patient in a screw-engagement manner. The implant 10 has a plurality of screw threads 10-2 formed on the outer circumferential surface thereof so as to be screwably coupled to the body of the patient, and a plurality of screw threads 10-4 formed on the inner circumferential surface of the upper wall thereof so as to allow the cover unit 20 to be screwably coupled to the screw threads 10-4.

Herein, although it has been described that the coupling structure of the implant 10 and the cover unit 20 is implemented in a screw-engagement manner, the coupling structure implemented in a sliding manner or a press-fit manner also falls within the technical scope of the present invention.

In addition, the accommodating part 12 is formed concavely in the implant 10 to provide a space for accommodating the drug cartridge 30 therein. The accommodating part 12 is formed in a shape corresponding to that of an outer appearance of the drug cartridge 30, particularly, is preferably formed in a cylindrical shape. But, the accommodating part 12 formed in a polygonal shape also falls within the technical scope of the present invention.

Moreover, at least one guide groove 12-5 is formed on the inner circumferential surface of the accommodating part 12 so as to guide the coupling and withdrawal of the drug cartridge 30 to and from the implant 10.

In addition, although it has been described herein that the guide groove 12-5 is formed on the inner circumferential surface of the accommodating part 12, the formation of a guide projection on the inner circumferential surface of the accommodating part 12 instead of the guide groove also falls within the technical scope of the present invention.

Also, the diffusion parts 14 are penetratingly formed sequentially on the circumferential surface of the accommodating part 12 in such a manner as to be spaced apart from each other at regular intervals. The diffusion part 14 provides a space allowing the content of the drug cartridge 30 to be diffused radially in a circumferential direction of the implant. Particularly, the diffusion part 14 may be formed to have various shapes and sizes in consideration of flowability of the content of the drug cartridge 30.

In addition, in the formation process of the diffusion part 14, the diffusion part 14 is formed as a diffusion hole which is gradually reduced or increased in cross-section as it goes toward an outlet thereof from an inlet thereof due to different cross sections of the inlet and the outlet of the diffusion part 14 as shown in FIG. 2(a).

In addition, the diffusion part 14 may be formed as an inclined hole which is gradually downwardly inclined as it goes toward the outlet thereof from the inlet thereof due to different gradients of the inlet and outlet of the diffusion part 14 as shown in FIG. 2(b). Alternatively, the diffusion part 14 may be formed as a horizontal hole which is uniform in the cross sections and the gradients of the inlet and the outlet thereof, respectively as shown in FIG. 2(c).

Of course, it is to be noted that in the formation process of the diffusion part 14, a combination of the method of forming the diffusion part 14 having different cross sections as shown in FIG. 2(a) and the method of forming the diffusion part 14 having different gradients as shown in FIG. 2(b) falls within the technical scope of the present invention.

Further, the injection parts 16 are formed sequentially on the outer circumferential surface of the implant 10 in such a manner as to be spaced apart from each other at regular intervals so that the injection parts 16 are brought into close contact with a medullary bone B. The injection parts 16 serve to dischargedly inject the content of the drug cartridge 30 into the human body.

In this case, the injection parts 16 can be formed on the crests or valleys of the screw threads 10-2 of the implant 10, but are preferably formed sequentially on the valleys of the screw threads 10-2 in consideration of the drug administration efficiency of the drug cartridge 30.

In the meantime, the cover unit 20 is coupled to the accommodating part 12 of the implant 10 to prevent leakage of the content of the drug cartridge 30 and block the foreign substances from being introduced into the accommodating space. The cover unit 20 is coupled to the upper inner portion of the accommodating part 12 of the implant 10 in a screw-engagement manner or in a press-fit manner.

In this case, in the formation process of the cover unit 20, the cover unit 20 preferably has a straight or cross-shaped screw groove 22 formed on the top surface of a head part thereof so as to maximize workabilities and convenience in the engagement and disengagement process of the cover unit 20 to and from the implant 20.

In addition, the drug cartridge 30 is selectively fixedly insert into the accommodating part 12. The drug cartridge 30 has at least one guide projection 32 formed on the outer circumferential surface thereof so as to be fittingly inserted into the guide groove 12-5 in a male-and-female mating manner.

Further, the drug cartridge 30 has a withdrawal lug 34 of a handgrip type formed on the top surface thereof so that the withdrawal lug is used to accommodate or withdraw the drug cartridge 30 in or from the accommodating space.

In this case, the drug cartridge 30 preferably includes an absorption member built therein to absorb a drug in a housing 36 thereof. In particular, the housing 36 preferably has a plurality of through-holes having intervals and sizes corresponding to those of the diffusion parts 14 to maximize the efficiency of drug delivery.

Of course, although it has been described herein that the absorption member is built in the housing 36 of the drug cartridge 30, a configuration in which the housing 36 is excluded also falls within the technical scope of the present invention.

Hereinafter, the operation of the drug delivery device according to the present invention will be described in detail.

First, in the case where it is desired to operate the drug delivery device according to the present invention, the drug delivery device 1 must be inserted and fastened into a medullary bone B via a cortical bone A of a patient with a chronic disease as shown in FIG. 3.

Subsequently, the drug cartridge 30 containing a medical component suitable for the treatment of a patient with a chronic disease is accommodated in the accommodating part 12 of the implant 10 in a state in which the cover unit 20 is removed from the implant 10 to cause the accommodating part to be opened, and then is fastened into the accommodating part 12 again.

In this process, the accommodation and withdrawal of the drug cartridge 30 in and from the accommodating part 12 of the implant 10 is smoothly performed by the interaction between the guide groove 12-5 formed on the implant 10 and the guide projection 32 formed on the drug cartridge 30

By doing so, as shown in FIG. 3, the injection parts 16 of the drug delivery device 1 are positioned in the medullary bone B of the patient with a chronic disease, and thus the content of the drug cartridge 30 is diffusively administered into the body of the patient by virtue of diffusion action due to osmosis.

On the other hand, in the case where the drug cartridge 30 accommodated in the accommodating part 12 is exhausted and thus it is desired to replace the drug cartridge with new one, the drug cartridge 30 can be withdrawn from implant 10 by pulling the withdrawal lug 34 out in a state in which the cover unit 20 is removed from the implant 10 to cause the accommodating part to be opened.

While the present invention has been described in connection with the exemplary embodiments illustrated in the drawings, they are merely illustrative embodiments, and the invention is not limited to these embodiments. It is to be understood that various equivalent modifications and variations of the embodiments can be made by a person having an ordinary skill in the art without departing from the spirit and scope of the present invention. Therefore, the true technical scope of the present invention should be defined by the technical spirit of the appended claims.

1. A drug delivery system using an implant comprises:
an implant 10 configured to be implanted into the human body to perform a support,
wherein the implant 10 includes an accommodating part 12 formed therein to allow a drug cartridge 30 to be accommodated therein, a plurality of diffusion parts 14 formed in the circumferential wall of the accommodating part 12 to allow the content of the drug cartridge 30 to be diffusively discharged to the outside of the implant therethrough, and a plurality of injection parts 16 formed at associated ones of distal ends of the diffusion units 14 to allow the content of the drug cartridge 30 to be injected into the human body therethrough; and
a cover unit 20 coupled to the implant 10 and configured to perform a closing function to open or close the accommodating part 12 of the implant 10.

2. The drug delivery system using an implant according to claim 1, wherein the implant 10 is mounted on an alveolar bone of a patient with a chronic disease.

3. The drug delivery system using an implant according to claim 1, wherein the cover unit 20 is coupled to the accommodating part of the implant 10 in a screw-engagement manner or in a press-fit manner.

4. The drug delivery system using an implant according to claim 1, wherein the cover unit 20 has a straight or cross-shaped screw groove 22 formed on the top surface of a head part thereof.

5. The drug delivery system using an implant according to claim 1, wherein the accommodating part 12 has a guide groove 12-5 is formed on the inner circumferential surface thereof so as to guide the coupling and withdrawal of the drug cartridge 30 to and from the implant 10.

6. The drug delivery system using an implant according to claim 1, wherein the drug cartridge 30 has a guide projection 32 formed on the outer circumferential surface thereof so as to be fittingly inserted into the guide groove 12-5 in a male-and-female mating manner.

7. The drug delivery system using an implant according to claim 1, wherein the drug cartridge 30 has a withdrawal lug 34 formed on the top surface thereof so that the withdrawal lug is used to easily accommodate or withdraw the drug cartridge 30 in or from the accommodating space.

8. The drug delivery system using an implant according to claim 1, wherein the diffusion part 14 is a diffusion hole formed such that the cross sections of an inlet and an outlet thereof are different from each other.

9. The drug delivery system using an implant according to claim 1, wherein the diffusion part 14 is an inclined hole formed such that the gradients of an inlet and an outlet thereof are different from each other.

10. The drug delivery system using an implant according to claim 1, wherein the diffusion part 14 is a horizontal hole formed such that the cross sections and the gradients of an inlet and an outlet thereof are the same as each other, respectively.
